(19) United States
(12) Patent Application Publication
(10) Pub. No.: US 2026/0321395 A1
(43) Pub. Date: Oct. 15, 2026

(54) Database matching using feature assessment

(71) Applicant: Siemens Medical Solutions USA, Inc., Malvern, PA (US)

(72) Inventors: Alexander Hans Vija, Evanston, IL (US); Francesc dAssis Massanes Basi, Chicago, IL (US)

(21) Appl. No.: 18/880,869

(22) PCT Filed: Oct. 6, 2022
(86) PCT No.: PCT/US2022/077640

§ 371 Date: Jan. 3, 2025

(51) Int. Cl. G16H 30/20; G06T 11/00; G06V 10/74; G16H 30/40; G16H 70/00

(52) U.S. Cl. CPC G16H 30/20; G06T 11/00; G06V 10/761; G16H 30/40; G16H 70/00

(57) ABSTRACT

A system and method includes determination of first image metrics of a first image of a phantom, the first image generated by a first imaging system based on first image formation parameters, and the first imaging system and first image formation parameters associated with a plurality of reference images, generation of a plurality of images of the phantom, each of the plurality of images generated using different respective image formation parameters, determination of second image metrics for each of the plurality of generated images, identification of one of the plurality of generated images based on the first image metrics and the second image metrics for each of the plurality of generated images, generation of an image of an object using the image formation parameters used to generate the identified one of the plurality of generated images, and comparison of the generated image of the object to one or more of the plurality of reference images.

### BACKGROUND

A “Normals” database may be used to assist the evaluation of medical images. A Normals database includes reference medical images of healthy subjects and/or subjects associated with a low likelihood of developing one or more diseases. Normals databases are used in cardiology and neurology but are not limited to these fields.

The reference images of the healthy/low-likelihood subjects stored within a Normals database are acquired using a particular imaging system, a particular imaging protocol, and a particular image data processing method. Accordingly, to use a Normals database, an image is acquired of a patient using the same imaging system, imaging protocol and processing method as was used to acquire the reference images of the Normals database. The image is then compared to reference images of the Normals Database. If the image differs from the reference images by more than a certain degree, for example, the image may be flagged as requiring clinical follow-up.

The reference images of a Normals database are therefore ideally only used to evaluate images which are acquired in the same manner as the reference images. If images are to be acquired using an imaging system, imaging protocol and processing method, any of which is different from those used to acquire the reference images of a Normals database, a new Normals database must be generated.

Generation of a Normals database is extremely costly. Accordingly, an imaging center which uses a particular Normals database may determine to not upgrade to a new imaging system or a new processing method because such an upgrade may render the particular Normals database unusable. Even if the imaging center decides to solicit creation of a new Normals database due to a change to its imaging chain, significant time may pass until the new database is available for use.

Systems are therefore desired to efficiently facilitate evaluation of medical images acquired using an imaging system, imaging protocol and processing method against a Normals database of reference images acquired using a different imaging system, imaging protocol and/or processing method.

### BRIEF DESCRIPTION OF THE DRAWINGS

FIG. 1 is a block diagram of a system to determine image formation parameters for an imaging system based on a reference imaging system and a phantom according to some embodiments;

FIG. 2 is a block diagram of a system to generate images for comparison to reference images according to some embodiments;

FIG. 3 is a flow diagram of a process to determine image formation parameters for an imaging system based on a reference imaging system and a phantom and to generate images using the image formation parameters for comparison to reference images according to some embodiments;

FIGS. 4A and 4B are views of a phantom according to some embodiments;

FIG. 5 is a graph of image resolution versus image noise for various image formation parameters of an imaging system according to some embodiments;

FIG. 6 is a block diagram of a system to convert reference images based on a target imaging system and a phantom according to some embodiments;

FIG. 7 is a block diagram of a system to generate images for comparison to converted reference images according to some embodiments;

FIG. 8 is a flow diagram of a process to convert reference images based on a target imaging system and a phantom and to generate images for comparison to converted reference images according to some embodiments; and

FIG. 9 is a view of an imaging system according to some embodiments.

### DETAILED DESCRIPTION

The following description is provided to enable any person in the art to make and use the described embodiments and sets forth the best mode contemplated for carrying out the described embodiments. Various modifications, however, will remain apparent to those in the art.

Some embodiments use a phantom to establish a relationship between a first imaging system and first image formation parameters and a second imaging system and second image formation parameters. The first imaging system and first image formation parameters may be associated with reference images of a Normals database, while the imaging system and second image formation parameters are to be used to acquire images which will be compared to the reference images. For purposes of the present description, image formation parameters include any parameters associated with acquisition of data (e.g., acquisition time, injection profile, sensitivity) and/or the reconstruction of an image from the acquired data (e.g., noise reduction, reconstruction algorithm, number of updates, post-smoothing).

In some embodiments, the second image formation parameters to be used by the second imaging system are determined based on a first image of a phantom acquired using the first imaging system and first image formation parameters and on second images of the same phantoms acquired using the second imaging system and various different second image formation parameters. The determined second image formation parameters may be those which result in images of the phantom having metric values which are most-similar to the metric values of the first image. The metric values may comprise noise and edge resolution, but embodiments are not limited thereto. An image of a patient may then be acquired by the second imaging system using the determined second image formation parameters and compared to the reference images.

In other embodiments, a first image of a phantom is acquired using the first imaging system and first image formation parameters and a second image of the same phantom is acquired using a second imaging system and second image formation parameters. Metric values of the first image and the second image are determined. Next, based on the metric values, a mapping is determined to convert the first image to an image having metric values similar to the second image. The mapping is applied to the reference images of the Normals database to generate a converted set of reference images which are associated with the second imaging system and second image formation parameters. An image of a patient may then be acquired by the second imaging system using the second image formation parameters and compared to the converted reference images.

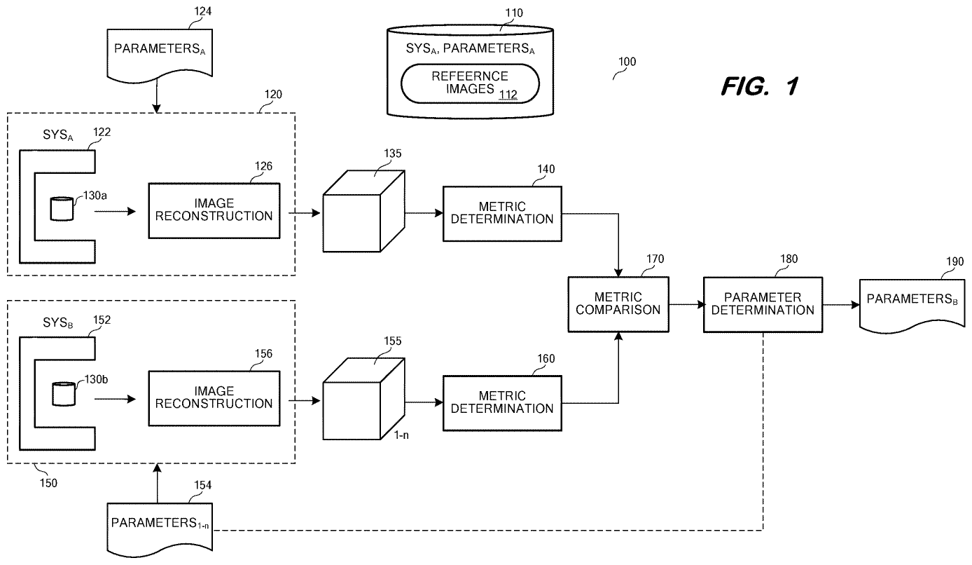

FIG. 1 illustrates system 100 according to some embodiments. Each component of system 100 and each other component described herein may be implemented using any combination of hardware and/or software. Some components may share hardware and/or software of one or more other components.

System 100 includes database 110 storing reference images 112. Database 110 may comprise a Normals database as described above but embodiments are not limited thereto. Reference images 112 comprise images acquired using a given model of an imaging system (i.e., Sys_{A} 122) and a particular set of image formation parameters (i.e., Parameters_{A} 124). As mentioned above, Parameters_{A} 124 may comprise any parameters used to control operation of Sys_{A} 122 to acquire data or based on which image reconstruction component 126 reconstructs an image from the acquired data. Sys_{A} 122 and image reconstruction component 126 will be collectively referred to herein as image formation system 120.

Embodiments are not limited to any particular imaging modality. For example, Sys_{A} 122 may comprise a single-photon emission computed tomography (SPECT) system, a positron emission tomography (PET) system, a magnetic resonance (MR) system, a computed tomography (CT) system, or any other system for generating medical images that is or becomes known. In some embodiments, Sys_{A} 122 comprises a particular model of an imaging system (e.g., Siemens Symbia Evo).

Sys_{B} 152 may comprise any type and model of imaging system. According to some embodiments, Sys_{B} 152 acquires images using a same imaging modality (e.g., SPECT) as Sys_{A} 122 but comprises a different model of imaging system. The model of Sys_{B} 152 and the model of Sys_{A} 122 may be produced by different companies or by the same company. In some embodiments, Sys_{A} 122 and Sys_{B} 152 are the same model of imaging system.

According to some embodiments, image formation system 120 generates image 135 of phantom 130a based on Parameters_{A} 124. Specifically, Sys_{A} 122 executes an imaging protocol based on any corresponding parameter values of Parameters_{A} 124 and image reconstruction component 126 reconstructs the resulting data based on any reconstruction or data processing parameter values (e.g., type of reconstruction, number of updates (i.e., iterations), post-smoothing level) of Parameters_{A} 124.

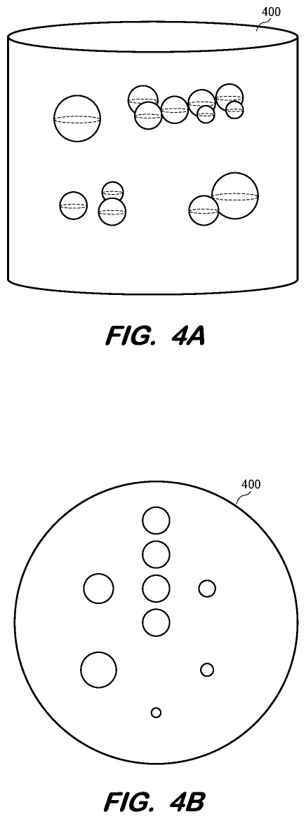

Phantom 130a may comprise any object visible to the imaging modality of Sys_{A} 122. An example of phantom 130a will be described with respect to FIGS. 4A and 4B but embodiments are not limited thereto. Phantom 130a may exhibit characteristics resulting in image 135 from which desired image metrics may be consistently determined using known techniques.

Metric determination component 140 determines one or more image metrics of image 135. The image metrics may include but are not limited to edge resolution, noise, uptake activity (in a case that the imaging modality of Sys_{A} 122 is molecular and phantom 130a includes photon-emitting radionuclides), and sphericity (in a case phantom 130a includes detectable spherical objects).

Similarly, but not necessarily contemporaneously, image formation system 150 generates images 155 of phantom 130b based on Parameters₁₋ₙ 154. In particular, and according to some embodiments, image formation system 150 generates N images 155, where each of the N images 155 is generated using a different set of Parameters₁₋ₙ 154. The different sets of Parameters₁₋ₙ 154 may vary in any number of manners. For example, a first plurality of sets of Parameters₁₋ₙ 154 may specify a first reconstruction algorithm and different respective post-smoothing levels, while a second plurality of sets of Parameters₁₋ₙ 154 may specify a second reconstruction algorithm and different respective post-smoothing levels.

Phantom 130b may be the same physical object as phantom 130a or a replica thereof. For example, phantoms 130a and 130b may comprise different objects but a same model of a phantom having identical dimensions and configurations. If phantom 130a was loaded with radionuclides prior to imaging by system 120, then phantom 130b is similarly-loaded prior to imaging by system 150.

Metric determination component 160 determines one or more image metrics of each of images 155. Next, metric comparison component 170 compares the metrics determined by metric determination component 140 to the metrics determined by metric determination component 160. The comparison may be intended to determine one of images 155 whose metrics are closest to the metrics determined for image 135. Parameter determination component 180 then determines which of Parameters₁₋ₙ 154 were used to acquire the determined one of images 155. The determined parameters are output as Parameters_{B} 190.

According to some embodiments, image formation system 150 generates one or more images 155 based on respective Parameters 154 and metric determination component 160 determines metrics thereof as described above. Metric comparison component 170 compares the metrics to the metrics to the metrics determined for image 135 and outputs the results of the comparison to parameter determination component 180. Contrary to the prior example, parameter determination component 180 generates one or more sets of parameters based on the comparison and image formation system 150 uses the one or more sets of parameters to generate one or more new images 155. Parameter determination component 180 may generate the one or more sets of parameters in an attempt to reduce the difference between the metrics determined for images 155 and the metrics determined for image 135. The foregoing process may continue until the difference between the metrics determined for a particular image 155 and the metrics determined for image 135 are within a threshold. Parameter determination component 180 outputs the parameters which were used to acquire the particular image 155 as Parameters_{B} 190.

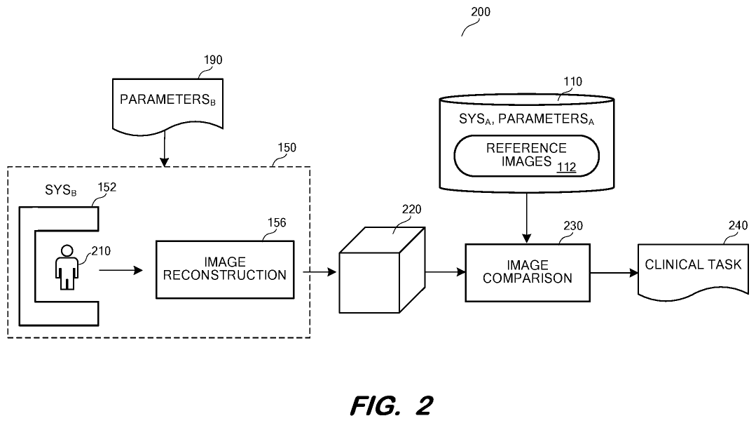

FIG. 2 is a block diagram of system 200 to generate images for comparison to reference images using parameters determined by system 100 according to some embodiments. For example, it is assumed that Parameters_{B} 190 were previously determined as described with respect to FIG. 1. As shown in FIG. 2, image formation system 150 of FIG. 1 generates image 220 of patient 210 based on Parameters_{B} 190. Image 220 may comprise an image of any portion of patient 210.

Due to the manner in which Parameters_{B} 190 were determined, image metrics of image 220 are assumed to be similar to image metrics of reference images 112 of database 110, thus facilitating their comparison. Accordingly, image comparison component 230 compares image 220 to reference images 112 using any known image comparison techniques and algorithms, including but not limited to visual comparison executed by a human.

Based on the comparison, image comparison component 230 may output clinical task 240. For example, if image 220 is not suitably similar to certain ones of reference images 112, image comparison component 230 may output clinical task 240 indicating further clinical tests to execute on patient 210. Embodiments may therefore facilitate usage of a Normals database containing reference images associated with a first imaging system and first image formation parameters to evaluate images generated using a second imaging system and second image formation parameters.

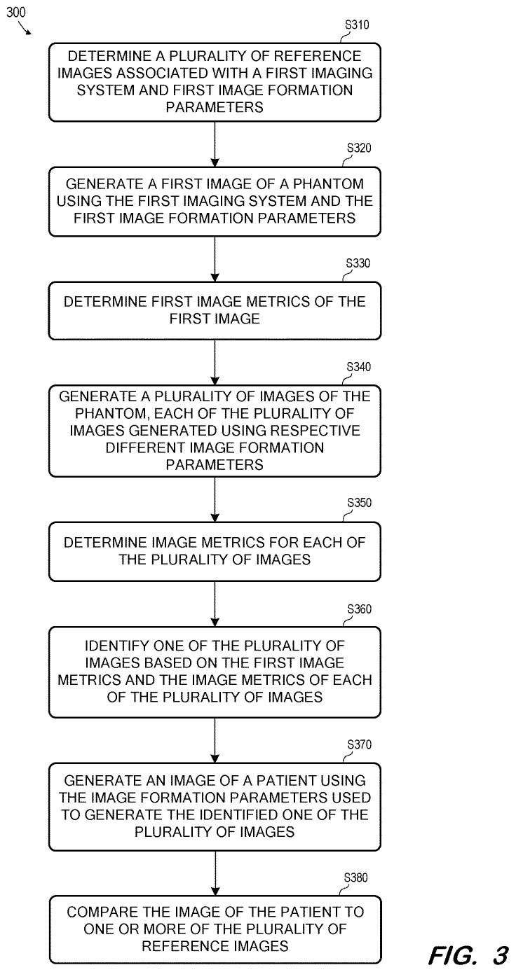

FIG. 3 is a flow diagram of process 300 to determine image formation parameters for an imaging system based on a reference imaging system and a phantom and to generate images using the image formation parameters for comparison to reference images according to some embodiments. In some embodiments, various hardware elements (e.g., one or more processing units such as one or more processors, one or more processor cores and one or more processor threads) execute program code to perform process 300. The steps of process 300 need not be performed by a single device or system, nor temporally adjacent to one another or in the order shown.

Process 300 and all other processes mentioned herein may be embodied in executable program code read from one or more of non-transitory computer-readable media, such as a disk-based or solid-state hard drive, a DVD-ROM, a Flash drive, and a magnetic tape, and then stored in a compressed, uncompiled and/or encrypted format. In some embodiments, hard-wired circuitry may be used in place of, or in combination with, program code for implementation of processes according to some embodiments. Embodiments are therefore not limited to any specific combination of hardware and software.

Process 300 may be executed by an imaging system vendor or an imaging center desiring to use a Normals database associated with a first imaging system and first image formation parameters to evaluate images generated using a second imaging system and second image formation parameters. As mentioned above, the first imaging system and the second imaging system may be a same imaging system model in some embodiments.

In some examples, S310-S330 may be executed by one entity (e.g., an entity possessing the first imaging system) while S340-S380 might be executed by another entity (e.g., an entity possessing the second imaging system). In still other examples, S310-S330 may be executed by one entity (e.g., an entity possessing the first imaging system), S340-S360 might be executed by another entity (e.g., an entity possessing the second imaging system), and S370-S380 might be executed by yet another entity (e.g., an entity using the second imaging system or a third imaging system of the same model as the second imaging system to acquire and evaluate patient images using the Normals database).

Initially, at S310, a plurality of reference images are determined. The plurality of reference images are associated with a first imaging system (e.g., a particular model of a SPECT imaging system) and first image formation parameters. The plurality of reference images may have each been acquired using the first imaging system and the first image formation parameters, and be stored in a Normals database as described above.

Next, at S320, a first image of a phantom is generated using the first imaging system and the first image formation parameters. Generation of the first image may include acquisition of multiple projection images and reconstruction of the projection images into a three-dimensional image. In some embodiments, more than one image is generated at S320.

The phantom may comprise any object visible to the first imaging system. FIG. 4A is a side view and FIG. 4B is a top view of phantom 400 according to some embodiments. Phantom 400 is cylindrical and includes thirteen hollow spheres into which may be loaded photon-emitting material, contrast agent, etc., depending on the imaging modality. Phantom 400 and its included spheres may be constructed from acrylic material but embodiments are not limited thereto. In some embodiments, one or more spheres is loaded with contrast agent and photon-emitting material, and other spheres are loaded with contrast agent and not with photon-emitting material. The remaining volume of phantom 400 may be filled with water and a suitable amount of photon-emitting material to achieve a desired background level of activity.

First image metrics of the first image are determined at S330. The determined first image metrics may include edge resolution, noise, uptake activity, and sphericity. Each of these metrics may be determined using known techniques. One method for determining noise and edge resolution is described in Vija, A. Hans, Siemens Healthineers, and Molecular Imaging Business Line. “xSPECT reconstruction method.” White Paper Order A91MI-10462 (2017): T1-7600, the contents of which are incorporated herein by reference for all purposes.

A plurality of images of the phantom are generated at S340. The phantom may be the same physical object as the phantom of S320 or a different instance of the same model of phantom having identical dimensions and configurations. The phantom imaged at S340 is preferably loaded with the same material at the same concentrations as the phantom imaged at S320.

Each of the plurality of images of the phantom is generated using image formation parameters which are different from the first image formation parameters. The plurality of images are generated using a second imaging system, which may or may not be the same model as the first imaging system. For example, the second imaging system may be the same model as the first imaging system but the reconstruction method specified by the different image formation parameters may be different from the reconstruction method specified by the first image formation parameters.

Image metrics are determined for each of the plurality of images at S350. The image metrics may be determined in the same manner as described above with respect to S330. Next, at S360, one of the plurality of images is identified based on the first image metrics and the image metrics of each of the plurality of images. For example, the first image metrics may be compared against each of the plurality of determined image metrics to identify a closest set of the plurality of determined image metrics. The one of the plurality of images which was generated using the closest set of the plurality of determined image metrics is identified at S360.

Since one or more image metrics may be determined for each image, identification of the closest set of image metrics may comprise determining a feature vector for each of the plurality of images. The feature vector for an image represents the values of the image metrics determined for the image. Certain metrics may be weighted differently from other metrics in the feature vector. Accordingly, S360 may comprise determining which of the feature vectors of the plurality of images is closest in feature space to the feature vector of the first image of the phantom.

In some embodiments, S360 includes a determination of whether an image accurately represents the phantom. In this regard, one or more of the metrics may be evaluated against expected metric values in view of a priori knowledge of the physical characteristics of the phantom. For example, an image which is associated with values of a shape deformation (or sphericity) metric, activity metric and/or a congruency metric which fall outside an expected range may be disqualified from consideration at S360, regardless of whether the values of other metrics (e.g., edge resolution and noise) of the image are closest to the corresponding metric values of the first image.

At S370, an image of a patient is generating using the image formation parameters which were used to generate the image identified at S360. By virtue of the preceding steps, it is assumed that image metrics of the generated image are similar to image metrics of the reference images determined at S310. Accordingly, the image of the patient is compared to one or more of the plurality of reference images at S380. A clinical task may be generated as a result of the comparison as described above.

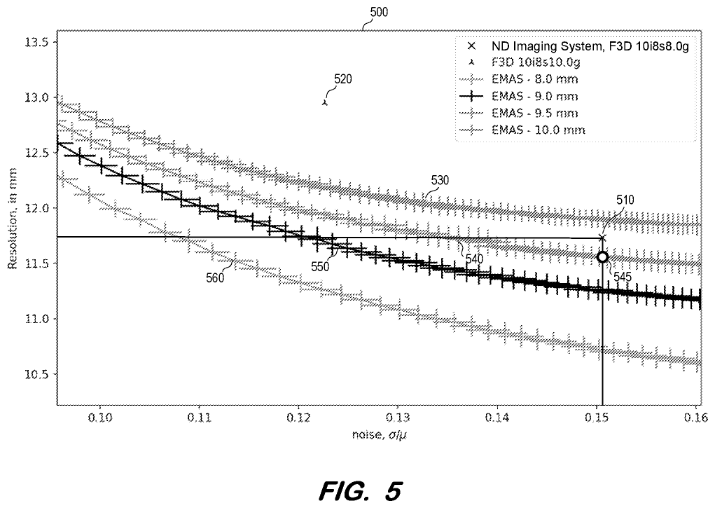

FIG. 5 depicts graph 500 of image resolution versus image noise for various image formation parameters of an imaging system according to some embodiments. In the present example, an image of a phantom was generated at S320 using an imaging system model (i.e., ND imaging System) and image formation parameters (i.e., Flash 3D (F3D) 10i8s8.0g) associated with a Normals database of reference images. Indicator 510 depicts values of the edge resolution and noise of the image as determined at S330.

It is assumed that it is desired to use the Normals database with a second, different, imaging system model. However, for explanatory purposes, indicator 520 indicates the edge resolution and noise of an image of the phantom acquired by the second imaging system model using the image formation parameters (i.e., F3D 10i8s8.0g) associated with the Normals database. Due to the differences in the metrics indicated by indicators 510 and 520, it would not be suitable to compare images acquired by the second imaging system model using the image formation parameters associated with the Normals database against the reference images of the Normals database.

Each of curves 530-560 represents the noise and edge resolution of images of the phantom acquired using the second imaging system model and a different respective set of image formation parameters (i.e., different reconstruction voxel sizes). Each vertical line of each curve represents metric values of an image generated using the image formation parameters of the curve and a different post-smoothing level. At certain post-smoothing levels, the image formation parameters associated with curves 540, 550 and 560 may generate images of the phantom having a same or better resolution than indicated by indicator 510 at lower noise levels.

Nevertheless, S360 includes a determination of a closest-matching set of resolution and noise metrics. It may be determined at S360 that point 545 of curve 540 is closest to indicator 510. Accordingly, the image generated using the image formation parameters associated with curve 540 and the post-smoothing level associated with point 545 is the image determined at S360. The image formation parameters associated with curve 540 and the post-smoothing level associated with point 545 are therefore used at S370 to generate an image of a patient for comparison to the reference images of the Normals database.

S340-S360 may be iterative in some embodiments. For example, one or more images of the phantom are generated at S340 using different image formation parameters, image metrics are determined for each image at S350, and it is determined at S360 whether any of the sets of metrics are acceptably close to the first image metrics of the first image. If so, it may also be determined whether the image metrics of the image corresponding to the closest set of metrics are physically accurate (e.g., with regard to shape deformation, activity and congruency).

If no metrics are acceptably close to the first image metrics of the first image, or if such close metrics are not physically accurate, flow returns to S340 to acquire another one or more images using different image formation parameters. The different image formation parameters may be determined based on differences between the previously-determined image metrics and the first image metrics. Flow proceeds from S360 to S370 once a set of metrics is identified which is acceptably close to the first image metrics of the first image and physically accurate.

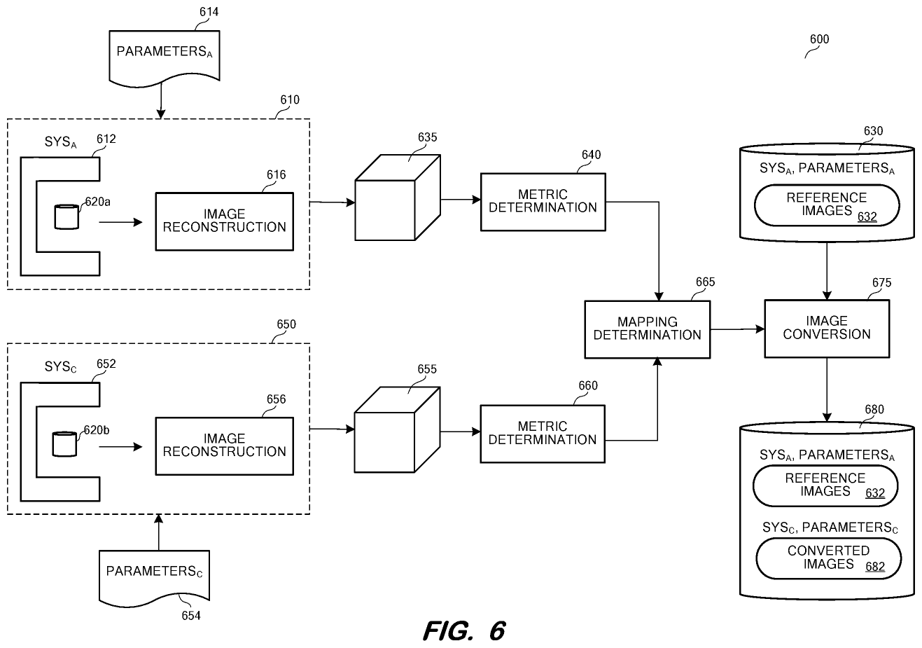

FIG. 6 is a block diagram of system 600 to convert reference images based on a target imaging system and a phantom according to some embodiments. System 600 includes database 630 storing reference images 632. Reference images 632 comprise images acquired using a given model of an imaging system (i.e., Sys_{A} 612) and a particular set of image formation parameters (i.e., Parameters_{A} 614). Parameters_{A} 614 may comprise any parameters used to control operation of Sys_{A} 612 to acquire data or based on which image reconstruction component 616 reconstructs an image from the acquired data. Sys_{A} 612 and image reconstruction component 616 are collectively referred to herein as image formation system 610.

Image formation system 610 generates image 635 of phantom 620a based on Parameters_{A} 614. Specifically, Sys_{A} 612 executes an imaging protocol based on any corresponding parameter values of Parameters_{A} 614 and image reconstruction component 616 reconstructs the resulting data based on any reconstruction or data processing parameter values (e.g., type of reconstruction, number of updates (i.e., iterations), post-smoothing level) of Parameters_{A} 614. Phantom 620a may be implemented as described with respect to phantom 130a and/or phantom 400, but embodiments are not limited thereto. Metric determination component 640 determines one or more image metrics of image 635 as described above.

Sys_{C} 652 may comprise any type and model of imaging system. Sys_{C} 652 may acquire images using a same imaging modality (e.g., SPECT) as Sys_{A} 612 but comprises a different model of imaging system. The model of Sys_{C} 652 and the model of Sys_{A} 612 may be produced by different companies or by the same company. In some embodiments, Sys_{A} 612 and Sys_{C} 652 are the same imaging system model.

Image formation system 650 generates images 655 of phantom 620b based on Parameters_{C} 654. Phantoms 620a and 620b may comprise different objects but a same model of a phantom having identical dimensions and configurations. If phantom 620a was loaded with radionuclides prior to imaging by system 120, then phantom 620b is similarly-loaded prior to imaging by system 150. Parameters_{C} 654 may comprise a default set of parameters, a preferred set of parameters for imaging system 652 in terms of image quality, speed, and/or patient region to be imaged, etc. That is, unlike the embodiments discussed with respect to FIGS. 1-3, Parameters_{C} 654 are “target” parameters to be used in conjunction with image formation system 650, rather than parameters to be determined based on considerations related to image formation system 610 or Parameters_{A} 614.

Metric determination component 660 determines one or more image metrics of image 655. Next, mapping determination component 665 determines a mapping based on the metrics determined by components 640 and 660. In one example, mapping determination component 665 determines a mapping which would result in image 635 exhibiting similar metric values as image 655. Such a mapping may specify application of a different reconstruction algorithm or different reconstruction parameters to the data acquired by Sys_{A} 614.

Image conversion component 675 applies the determined mapping to reference images 632 to generate converted images 682. Converted images 682 may be stored in updated Normals database 680 along with reference images 632. As illustrated in updated Normals database 680, converted images 682 are associated with Sys_{C} 652 and Parameters_{C} 654.

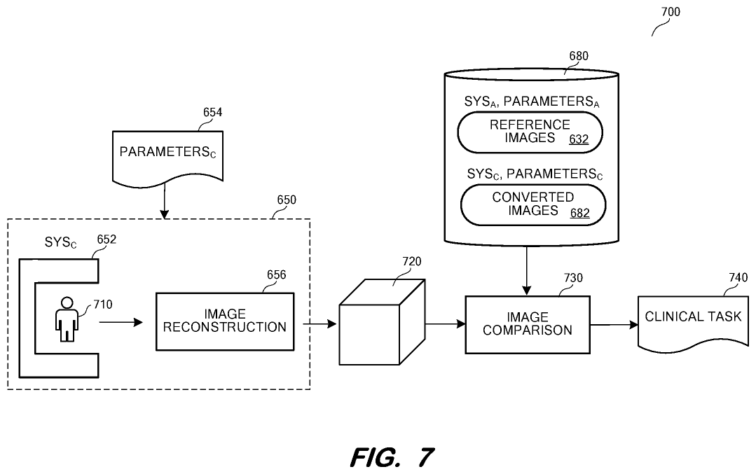

FIG. 7 is a block diagram of system 700 to generate images for comparison to reference images converted by system 600 according to some embodiments. As shown, image formation system 650 generates image 720 of patient 710 based on Parameters_{C} 654. Image 720 may comprise an image of any portion of patient 710.

Image comparison component 730 compares image 720 to converted images 682 using any known image comparison techniques and algorithms, including but not limited to visual comparison executed by a human. Image comparison component 730 may output clinical task 740 based on the comparison. Embodiments as depicted in FIGS. 6 and 7 may therefore also facilitate usage of a Normals database containing reference images associated with a first imaging system and first image formation parameters to evaluate images generated using a second imaging system and second image formation parameters.

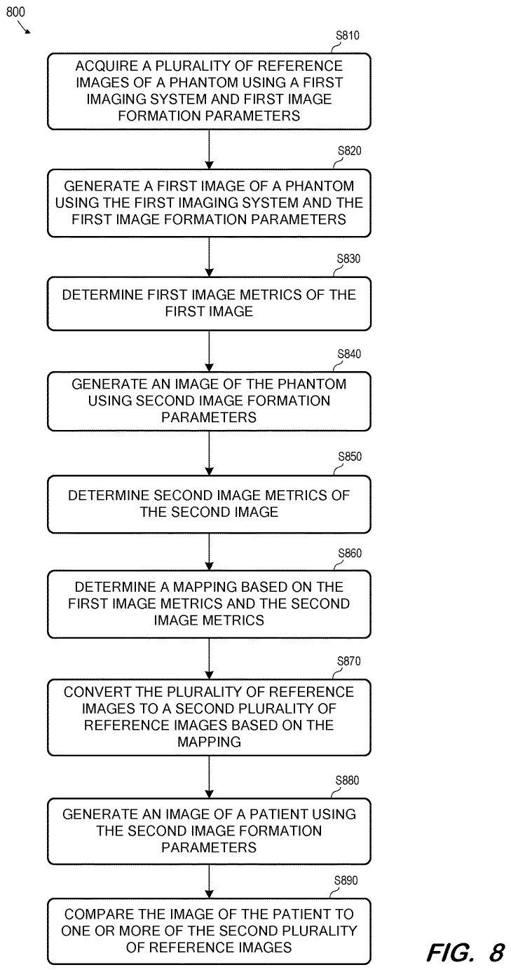

FIG. 8 is a flow diagram of process 800 to convert reference images based on a target imaging system and a phantom and to generate images for comparison to converted reference images according to some embodiments. Process 800 may be executed by an imaging system vendor or an imaging center desiring to use a Normals database associated with a first imaging system and first image formation parameters to evaluate images generated using a second imaging system and second image formation parameters. As mentioned above, the first imaging system and the second imaging system may be a same imaging system model in some embodiments.

S810-S830 may proceed as described above with respect to S310-S330 of process 300. Next, at S840, a second image of the phantom is generated using image formation parameters which are different from the first image formation parameters used at S820. The second image is generated using a second imaging system, which may or may not be the same model as the first imaging system used at S820. For example, the second imaging system may be the same model as the first imaging system but the reconstruction method specified by the different image formation parameters may be different from the reconstruction method specified by the first image formation parameters.

Image metrics are determined for the second image at S850. Next, at S860, a mapping is determined based on the first image metrics and the second image metrics. In some embodiments, S860 comprises determination of re-processing steps to apply to the first image such that the re-processed first image exhibits image metrics suitably close to the image metrics of the second image. The re-processing steps may comprise a reconstruction algorithm including a particular number of updates and/or post-smoothing level. The determination at S860 may include re-processing the first image in several different manners and choosing the re-processing steps which generated an image which exhibits image metrics closest to the image metrics of the second image.

Each of the plurality of reference images is converted based on the determined mapping at S870. The second imaging system generates an image of a patient at S880 using the second image formation parameters. S880 may occur at a different time and location than S870 and the second imaging system of S880 may comprise a different instance but a same model as the second imaging system of S870. It can be assumed that image metrics of the image generated at S880 are similar to image metrics of the reference images converted at S870. The image of the patient is compared to one or more of the plurality of converted reference images at S890, possibly resulting in generation of a clinical task.

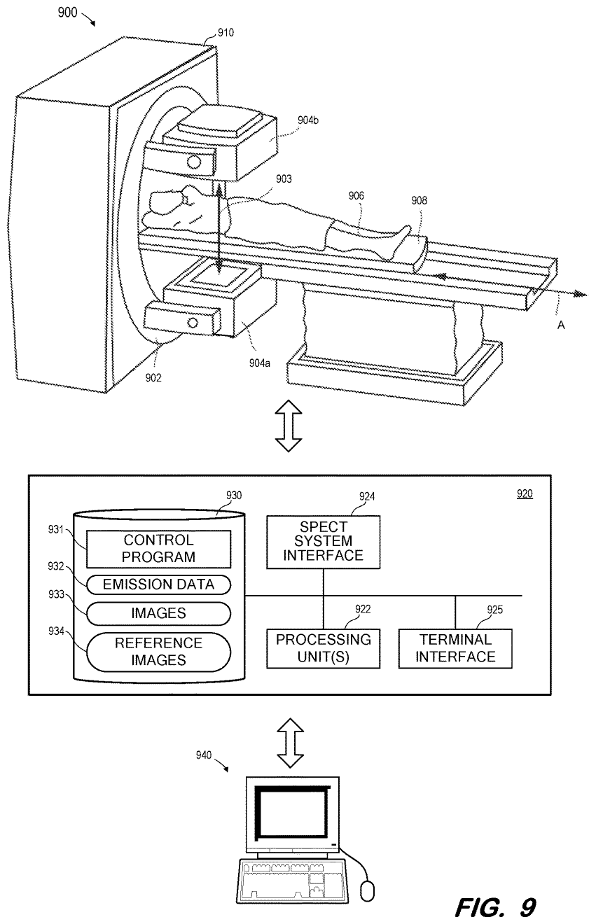

FIG. 9 illustrates imaging system 900 according to some embodiments. System 900 is a SPECT imaging system as is known in the art, but embodiments are not limited thereto. Each component of system 900 may include other elements which are necessary for the operation thereof, as well as additional elements for providing functions other than those described herein.

System 900 includes housing 910 for gantry 902 to which two or more gamma cameras 904a, 904b are attached, although any number of gamma cameras can be used. A detector within each gamma camera detects gamma photons (i.e., emission data) emitted by a radioactive tracer injected into the body of patient 906 lying on bed 908. Bed 908 is slidable along axis-of-motion A. At respective bed positions (i.e., imaging positions), a portion of the body of patient 906 is positioned between gamma cameras 904a, 904b in order to capture emission data from that body portion from various projection angles.

Control system 920 may comprise any general-purpose or dedicated computing system. Control system 920 includes one or more processing units 922 configured to execute executable program code to cause system 920 to operate as described herein, and storage device 930 for storing the program code. Storage device 930 may comprise one or more fixed disks, solid-state random access memory, and/or removable media (e.g., a thumb drive) mounted in a corresponding interface (e.g., a USB port).

Storage device 930 stores program code of control program 931. One or more processing units 922 may execute control program 931 to, in conjunction with SPECT system interface 924, control motors, servos, and encoders to cause gamma cameras 904a, 904b to rotate along gantry 902 and to acquire two-dimensional emission data 932 at defined imaging positions during the rotation.

Control program 931 may further be executed to reconstruct images 933 based on specified parameters. The specified parameters may have been determined as described with respect to FIGS. 1 and 3 so as to facilitate the comparison of images 933 with reference images 934. In other embodiments, reference images 934 include reference images which have been converted based on the specified parameters of system 900 as described above with respect to FIGS. 6 and 8.

Terminal 940 may comprise a display device and an input device coupled to terminal interface 925 of system 920. Terminal 940 may receive and display images 933 and reference images 934, as well as a user interface to assist comparisons therebetween. In some embodiments, terminal 940 is a separate computing device such as, but not limited to, a desktop computer, a laptop computer, a tablet computer, and a smartphone.

Those in the art will appreciate that various adaptations and modifications of the above-described embodiments can be configured without departing from the claims. Therefore, it is to be understood that the claims may be practiced other than as specifically described herein.

What is claimed is:

1. A method comprising:
determining first image metrics of a first image of a phantom, the first image generated by a first imaging system based on first image formation parameters, and the first imaging system and first image formation parameters associated with a plurality of reference images;
generating a plurality of images of the phantom, each of the plurality of images generated using different respective image formation parameters;
determining second image metrics for each of the plurality of generated images;
identifying one of the plurality of generated images based on the first image metrics and the second image metrics for each of the plurality of generated images;
generating an image of an object using the image formation parameters used to generate the identified one of the plurality of generated images; and
comparing the generated image of the object to one or more of the plurality of reference images.

2. A method according to claim 1, wherein the first image metrics comprise edge resolution and noise, and the second image metrics comprise edge resolution and noise.

3. A method according to claim 2, wherein the first image metrics comprise uptake activity and sphericity, and the second image metrics comprise uptake activity and sphericity.

4. A method according to claim 3, wherein identifying one of the plurality of generated images comprises identifying one of the plurality of generated images associated with second image metrics which are closest to the first image metrics.

5. A method according to claim 2, wherein identifying one of the plurality of generated images comprises identifying one of the plurality of generated images associated with second image metrics which are closest to the first image metrics.

6. A method according to claim 1, wherein the plurality of images of the phantom are generated using a second imaging system and the image of the object is generated using the second imaging system.

7. A method comprising:
determining first image metrics of a first image of a phantom, the first image generated by a first imaging system based on first image formation parameters, and the first imaging system and first image formation parameters associated with a plurality of reference images;
generating a second image of the phantom using a second imaging system and second image formation parameters;
determining second image metrics of the second image;
determining a mapping based on the first image metrics and the second image metrics;
converting each of the plurality of reference images to a second plurality of reference images based on the mapping;
generating an image of an object using the second imaging system and the second image formation parameters; and
comparing the generated image of the object to one or more of the converted plurality of reference images.

8. A method according to claim 7, wherein the first image metrics comprise edge resolution and noise, and the second image metrics comprise edge resolution and noise.

9. A method according to claim 8, wherein the first image metrics comprise uptake activity and sphericity, and the second image metrics comprise uptake activity and sphericity.

10. A method according to claim 8, wherein determining a mapping comprises determining reconstruction parameters based on the first image metrics and the second image metrics.

11. A method according to claim 7, further comprising:
storing the converted plurality of reference images with the plurality of reference images.

12. A method according to claim 7, further comprising:
generating a third image of the phantom using a third imaging system and third image formation parameters;
determining third image metrics of the third image;
determining a second mapping based on the first image metrics and the third image metrics;
converting each of the plurality of reference images to a second converted plurality of reference images based on the second mapping; and
storing the second converted plurality of reference images with the converted plurality of reference images and the plurality of reference images.

13. A non-transitory computer-readable medium storing program code executable by a processing unit to:
determine first image metrics of a first image of a phantom, the first image generated by a first imaging system based on first image formation parameters, and the first imaging system and first image formation parameters associated with a plurality of reference images;
generate a plurality of images of the phantom, each of the plurality of images generated using different respective image formation parameters;
determine second image metrics for each of the plurality of generated images;
identify one of the plurality of generated images based on the first image metrics and the second image metrics for each of the plurality of generated images;
generate an image of an object using the image formation parameters used to generate the identified one of the plurality of generated images; and
compare the generated image of the object to one or more of the plurality of reference images.

14. A medium according to claim 13, wherein the first image metrics comprise edge resolution and noise, and the second image metrics comprise edge resolution and noise.

15. A medium according to claim 14, wherein the first image metrics comprise uptake activity and sphericity, and the second image metrics comprise uptake activity and sphericity.

16. A medium according to claim 15, wherein identification of one of the plurality of generated images comprises identification of one of the plurality of generated images associated with second image metrics which are closest to the first image metrics.

17. A medium according to claim 15, wherein identification of one of the plurality of generated images comprises identification of one of the plurality of generated images associated with second image metrics which are closest to the first image metrics.

18. A medium according to claim 13, wherein the plurality of images of the phantom are generated using a second imaging system and the image of the object is generated using the second imaging system.
